(19) United States
(12) Patent Application Publication
(10) Pub. No.: US 2024/0407902 A1
(43) Pub. Date: Dec. 12, 2024

(54) Dynamic Joint Alignment Brace For An Animal

(71) Applicants: Kristen Ruth Jacobs, Lake Oswego, OR (US); David Whitt Sagan, Lake Oswego, OR (US)

(72) Inventors: Kristen Ruth Jacobs, Lake Oswego, OR (US); David Whitt Sagan, Lake Oswego, OR (US)

(21) Appl. No.: 18/741,639

(22) Filed: Jun. 12, 2024

Related U.S. Application Data
(60) Provisional application No. 63/472,417, filed on Jun. 12, 2023.

(51) Int. Cl. A61D 9/00; A61F 5/01

(52) U.S. Cl. CPC A61D 9/00; A61F 5/0193; A61F 2005/0179

(57) ABSTRACT

A dynamic joint alignment brace for an animal that is particularly suitable for reducing symptoms associated with canine hip dysplasia (CHD) and other musculoskeletal conditions that may be common in animals with a skeletal structure including canines and the like. The dynamic joint alignment brace applies a force to the animal's leg(s) in a direction that improves alignment of the leg bone(s) at the affected joint(s) throughout the range of motion of the leg(s). In disclosed embodiments, a resilient biasing structure, which is biased into a non-neutral position when worn, provides a force that persuades the animal's leg(s) into alignment with its respective joint(s).

### CROSS-REFERENCE TO RELATED APPLICATION

This application claims priority to U.S. Provisional Patent Application Ser. No. 63/472,417, filed on Jun. 12, 2023, the disclosure of which is hereby incorporated by reference.

### BACKGROUND OF THE INVENTION

Hip dysplasia is an improper formation of the hip joint(s) that occurs during growth and results in laxity (looseness) of the hip joint(s) followed by degenerative arthritic change in the hip joint(s). To facilitate understanding, an exemplar healthy canine hip joint is shown in FIG. 4 (PRIOR ART) and an exemplar canine hip joint experiencing hip dysplasia is shown in FIG. 5 (PRIOR ART). Hip dysplasia is a genetic disease in canines that is affected by factors such as diet, environment, exercise, growth rate, muscle mass, other musculoskeletal conditions, and or trauma. Symptoms of hip dysplasia are pain, limping, and or lameness as a result of chronic subluxation (partial dislocation), and or luxation (complete dislocation), and or degenerative arthritic changes of the hip joint(s). Additionally, a canine with chronic hip dysplasia will often have both the underdevelopment of the rear-limb musculature and the overdevelopment of the forelimb and shoulder musculature resulting from the avoidance of movements that may be painful.

Treatment of canine hip dysplasia (CHD), and other musculoskeletal conditions that may be common in animals with a skeletal structure including canines and the like, may depend upon a canine's age, factors affecting the condition, and or severity of the condition when CHD is accurately diagnosed. If detected early, the effects of CHD may be mitigated by conservative treatments which may include but are not limited to lifelong weight management strategies such as diet modifications and appropriate exercise in addition to supplementation with omega-3 fatty acids, glucosamine, and chondroitin. As with any joint disease, movement may be painful and therefore a canine may become lethargic even in the early stages of CHD.

Current solutions in the field such as the example shown in FIG. 6 (PRIOR ART) may only address direct compression of the hip joint(s) which may limit mobility and may lead to weakening and or improper alignment of the associated tissues (ligaments, tendons, and muscles) around and below the hip joint(s). Similarly, other known structures, such as those shown in published U.S. patent application Ser. No. 17/422,843/Publication No. US2022/0117715 to Mills and published French Patent Application No. 8008242/Publication Number FR2480109 to Perot, attempt to rigidly force and hold the animal's legs in abduction, thereby limiting the range of movement of the animal and compromising the ability of the animal to easily assume and move between standing, sitting and laying positions.

More advanced stages of CHD may require prescription pain-relieving medication and or surgery, even in severe cases, preventing chronic subluxation and or luxation and improving muscle mass and tone may mitigate or halt degenerative arthritic changes of the hip joint(s).

As with any medical condition it may be the objective of a clinician to minimize the necessity for medication and or surgery whenever possible. However, the least invasive way to accomplish this goal may be unattractive to a canine due to hip pain. It has therefore been a long-standing desire for a device which may alleviate pain associated with CHD. Minimizing pain may allow for more conservative treatment to be pursued and consequently may eliminate the need for prescription pain-relieving medication and or surgery.

### SUMMARY OF THE INVENTION

The present exemplary embodiments provide a dynamic joint alignment brace for an animal that uses at least one resilient member deflected from its neutral position when operably secured to the animal to urge an animal's joint engagement toward a more desirable orientation without unduly compromising the freedom of movement of the animal. In addition, a plurality of resilient members, each with different resiliencies, may be operably secured to the animal's leg thereby allowing the direction of the force urging the leg into abduction to be optimized and fine-tuned for the animal.

In one disclosed embodiment the dynamic joint alignment brace may be particularly suitable for use in the treatment of canine hip dysplasia (CHD) for relief of symptoms associated therewith.

Preferred embodiments of the dynamic joint alignment brace may include the resilient member(s) having a neutral position and being deflected from its neutral position when operably secured to the animal's leg thereby flexibly urging the leg toward joint abduction.

If desired, the resilient member(s) may extend to at least one complementary rear leg encircling object, such as a left rear leg encircling object 50 and a right rear leg encircling object 52 wherein a left rear leg encircling object 50 may include a left leg strap or cuff and may be adjustably sized (for example using hook and loop fastener) to partially or fully encircle the left leg of a canine about the thigh region 81 (FIGS. 1-3 (PRIOR ART)), and a right rear leg encircling object 52 may include a right leg strap or cuff and may be adjustably sized (for example using hook and loop fastener) to partially or fully encircle the right leg of an animal about the thigh region 81 (FIGS. 1-3 (PRIOR ART)).

The left rear leg encircling object 50 and a right rear leg encircling object 52 may be made of any material with particular attention paid to areas that may chafe and or cause discomfort. For example, a left rear leg encircling object 50 and a right rear leg encircling object 52 may be made of a soft resilient material such as neoprene with a hook and loop convention for donning and removing. Alternatively, a left rear leg encircling object 50 and a right rear leg encircling object 52 may be made of a non-textile type material such as molded plastic preferably with some level of added cushioning and made removable by durable hardware. The left rear leg encircling object 50 and right rear leg encircling object 52 may be detachably secured to resilient member 54 to apply a force that may persuade a canine's leg(s) laterally towards a more stable position where a canine's rear stance may be wider.

The dynamic joint alignment brace may serve as a support and alignment device principally suitable for decreasing the symptoms associated with CHD. Especially notable is hip joint pain resulting from chronic subluxation and or luxation due to hip joint laxity as shown in FIG. 5 (PRIOR ART) and or degenerative arthritic changes of the hip joint(s). By incorporating a force directed laterally, outward from the median plane of a canine, on the limb(s) below the affected hip joint(s) as shown in FIG. 9, incidents of subluxation and or luxation may be reduced or eliminated and may mitigate or halt degenerative arthritic changes of the hip joint(s) and may reduce or eliminate pain associated with CHD.

The dynamic joint alignment brace may provide a treatment device for CHD which may be utilized with or without medical supervision and which may mitigate or halt degenerative arthritic changes of the hip joint(s) prior to any necessary surgical intervention and may reduce or eliminate the need for prescription pain-relieving medication that may have negative health effects and accordingly would require ongoing blood panel screenings.

An additional possible feature of the dynamic joint alignment brace is that it may apply a constant and or adjustable force to the limb(s) of a canine which may continually correct the limb position in order to keep it in an orientation in which it is least likely to result in subluxation and or luxation of the hip joint(s).

Also, the dynamic joint alignment brace may offer increased animal mobility shown in FIG. 8 as compared to other more restrictive and or immobilizing devices available in the field such as those shown in FIG. 6 (PRIOR ART). Direct compression of the hip joint(s) may be useful for short-term treatment to prevent subluxation and or luxation of the hip joint(s), however, long-term treatment utilizing restrictive and or immobilizing devices as shown in FIG. 6 (PRIOR ART) may lead to weakening and or improper alignment of the associated tissues (ligaments, tendons, and muscles) around and below the hip joint(s) and may consequently result in an increased likelihood of subluxation and or luxation of the hip joint(s) and may subsequently require progressively increasing direct compression of the hip joint(s) to prevent subluxation and or luxation of the hip joint(s). A dynamic joint alignment brace 100 as shown in FIGS. 7-9 may provide increased mobility and may consequently lead to proper therapeutic strengthening and or proper alignment of the associated tissues (ligaments, tendons, and muscles) around and below the hip joint(s) and may subsequently reduce and or eliminate incidents of subluxation and or luxation of the hip joint(s). Additionally, a dynamic joint alignment brace 100 may improve the underdevelopment of the rear-limb musculature and the overdevelopment of the forelimb and shoulder musculature, promoting a healthy balance of the entire musculature system of a canine and may consequently improve a canine's quality of life and longevity.

Disclosed embodiments of the dynamic joint alignment brace also provide a canine's hip joint(s) stability without contact on or near the hip joint(s) as compared to other more restrictive and or immobilizing devices available in the field as shown in FIG. 6 (PRIOR ART). Direct compression of the hip joint(s) shown in FIG. 6 (PRIOR ART) may restrict circulation and may consequently result in edema and or damage of the limb(s) and or joint(s). Compression fabric and material utilized in restrictive and or immobilizing devices shown in FIG. 6 (PRIOR ART) may inhibit airflow and harbor bacteria and may consequently result in the development of skin abrasions, sores, and or infections. The dynamic joint alignment brace 100 may substantially decrease the number of contact points and or surfaces in direct contact with a canine's hip joint(s), limb(s), and skin, which may allow unrestricted circulation and may maintain the health of a canine's hip joint(s), limb(s), and skin.

The dynamic joint alignment brace 100 may also allow a canine freedom of movement without assistance. The unrestrictive properties of the dynamic joint alignment brace 100 may allow a canine to sit, lay down, sleep, and stand up independently from any surface, for example hardwood flooring, carpet, tile and more compliant or unstable surfaces such as bedding and the like. As the various components are arranged in use, the dynamic joint alignment brace 100 may allow a canine to perform its necessary biological activities unencumbered without the need for removal of the dynamic joint alignment brace 100, and without the likelihood soiling the dynamic joint alignment brace 100.

Other features and advantages of the subject disclosure will be apparent from the following more detailed description of the exemplary embodiments, taken in conjunction with the accompanying drawings which illustrate, by way of example, the principles of the subject disclosure.

### BRIEF DESCRIPTION OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the invention is not limited to the precise arrangements and instrumentalities shown.

In the drawings:

FIG. 1 (PRIOR ART) is a top view of a healthy canine skeletal structure.

FIG. 2 (PRIOR ART) is a left side view of the healthy canine skeletal structure of FIG. 1.

FIG. 3 (PRIOR ART) is a rear view of the healthy canine skeletal structure of FIG. 1.

FIG. 4 (PRIOR ART) is an enlarged fragmentary, isometric rear view of a healthy canine hip joint.

FIG. 5 (PRIOR ART) is an enlarged fragmentary, isometric rear view of the canine hip joint of FIG. 4 demonstrating hip dysplasia.

FIG. 6 (PRIOR ART) is a left side view of an exemplar known canine restrictive brace.

FIG. 7 is a top view of a canine 49 wearing a dynamic joint alignment brace 100 in accordance with a first embodiment 100A of the disclosed invention.

FIG. 8 is a left side view of the canine 49 wearing the dynamic joint alignment brace 100 of FIG. 7.

FIG. 9 is a rear view of the canine 49 wearing the dynamic joint alignment brace 100 of FIG. 7 and demonstrating corrected leg position caused by the canine 49 wearing the dynamic joint alignment brace 100.

FIG. 10 is a rear, isometric view of the dynamic joint alignment brace 100 of FIG. 7 showing a possible neutral position of a resilient member 54.

FIG. 11 is a rear, isometric view of the dynamic joint alignment brace 100 of FIG. 10 showing a possible deflected position of the resilient member 54 from the neutral position shown in FIG. 10.

FIG. 12 is a right, rear, isometric view of the dynamic joint alignment brace 100 of FIG. 7 showing the resilient member 54 in the possible deflected position of FIG. 11 when worn by a canine 49.

FIG. 13 is a top view of a canine 49 wearing a dynamic joint alignment brace 100 in accordance with a second embodiment 100B of the disclosed invention.

FIG. 14 is a left side view of the canine 49 wearing the dynamic joint alignment brace 100 of FIG. 13.

FIG. 15 is a rear view of the canine 49 wearing the dynamic joint alignment brace 100 of FIG. 13.

FIG. 16 is a right, side isometric view of the dynamic joint alignment brace 100 of FIG. 13 as applied to a canine 49.

FIG. 17 is a left, side isometric view of a canine 49 wearing a dynamic joint alignment brace 100 in accordance with a third embodiment 100C of the disclosed invention.

FIG. 18 is a top view of a canine 49 wearing a dynamic joint alignment brace 100 in accordance with a fourth embodiment 100D of the disclosed invention.

FIG. 19 is a left side view of the canine 49 wearing the dynamic joint alignment brace 100 of FIG. 18.

FIG. 20 is a rear view of the canine 49 wearing the dynamic joint alignment brace 100 of FIG. 18.

FIG. 21 is a top view of a canine 49 wearing a dynamic joint alignment brace 100 in accordance with a fifth embodiment 100E of the disclosed invention.

FIG. 22 is a left side view of the canine 49 wearing the dynamic joint alignment brace 100 of FIG. 21.

FIG. 23 is a rear view of the canine 49 wearing the dynamic joint alignment brace 100 of FIG. 21.

FIG. 24 is a rear, exploded view of the dynamic joint alignment brace 100 of FIG. 21.

FIG. 25 is a right, isometric view of the dynamic joint alignment brace 100 of FIG. 21 as applied to a canine 49.

FIG. 26 is an isometric view of a partial portion of a dynamic joint alignment brace 100 in accordance with a sixth embodiment 100F of the present invention.

FIG. 27 is a front isometric view of the partial portion of the dynamic joint alignment brace 100 of FIG. 26 showing possible alternative combination of elements.

FIG. 28 is a rear, isometric exploded view of the dynamic joint alignment brace 100 of FIG. 26.

FIG. 29 is a front, isometric view of a position of the dynamic joint alignment brace 100 of FIG. 26 showing a resilient member 54 deflected to a possible non-neutral position.

FIG. 30 is a side isometric view of the dynamic joint alignment brace 100 of FIG. 26 as applied to a canine 49 with the resilient member 54 in the possible non-neutral position shown in FIG. 29.

FIG. 31 is an isometric view of a partial portion of a dynamic joint alignment brace 100 showing a plurality of resilient members 54′, 54″ accordance with a seventh embodiment 100G of the present invention.

FIG. 32 is a front isometric view of the partial portion of the dynamic joint alignment brace 100 of FIG. 31 showing possible alternative combination of elements.

FIG. 33 is a top view of a canine 49 wearing a dynamic joint alignment brace 100 in accordance with an eighth embodiment 100H of the disclosed invention.

FIG. 34 is a left side view of the canine 49 wearing the dynamic joint alignment brace 100 of FIG. 33.

FIG. 35 is a rear view of the canine 49 wearing the dynamic joint alignment brace 100 of FIG. 33.

FIG. 36 is a top view of a partial portion of the dynamic joint alignment brace 100 of FIG. 33 showing a plurality of resilient members 54′, 54″.

FIG. 37 is a left side view of the partial portion of the dynamic joint alignment brace 100 of FIG. 36.

FIG. 38 is a rear view of the partial portion of the dynamic joint alignment brace 100 of FIG. 36.

FIG. 39 is a right side, front isometric view of the dynamic joint alignment brace 100 of FIG. 33.

FIG. 40 is a right side, rear isometric view of the dynamic joint alignment brace 100 of FIG. 33.

FIG. 41 is a right side, front isometric view of the dynamic joint alignment brace 100 of FIG. 33 as applied to a canine 49.

FIG. 42 is a top view of a canine 49 wearing a dynamic joint alignment brace 100 in accordance with a ninth embodiment 100I of the disclosed invention.

FIG. 43 is a left side view of the canine 49 wearing the dynamic joint alignment brace 100 of FIG. 42.

FIG. 44 is a rear view of the canine 49 wearing the dynamic joint alignment brace 100 of FIG. 42.

FIG. 45 is a top view of a canine 49 wearing a dynamic joint alignment brace 100 in accordance with a ninth embodiment 100J of the disclosed invention.

FIG. 46 is a left side view of the canine 49 wearing the dynamic joint alignment brace 100 of FIG. 45.

FIG. 47 is a rear view of the canine 49 wearing the dynamic joint alignment brace 100 of FIG. 45.

FIG. 48 is an image of Laika, a loyal and beloved friend and the inspiration for this invention, wearing the dynamic joint alignment brace 100 while resting comfortably in a laying position.

### DETAILED DESCRIPTION OF THE INVENTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting.

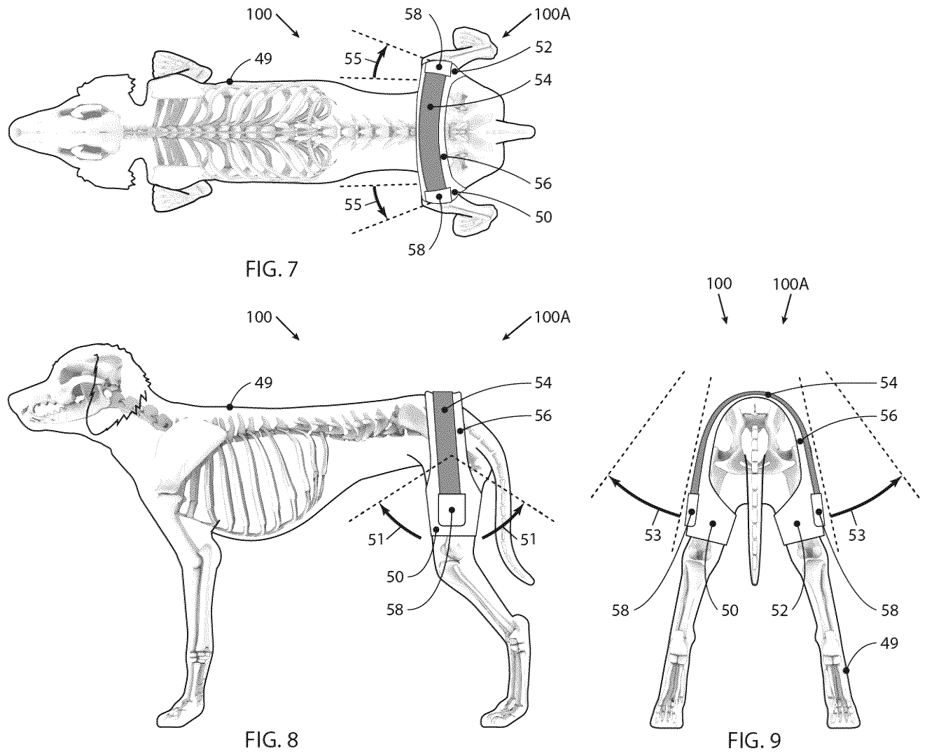

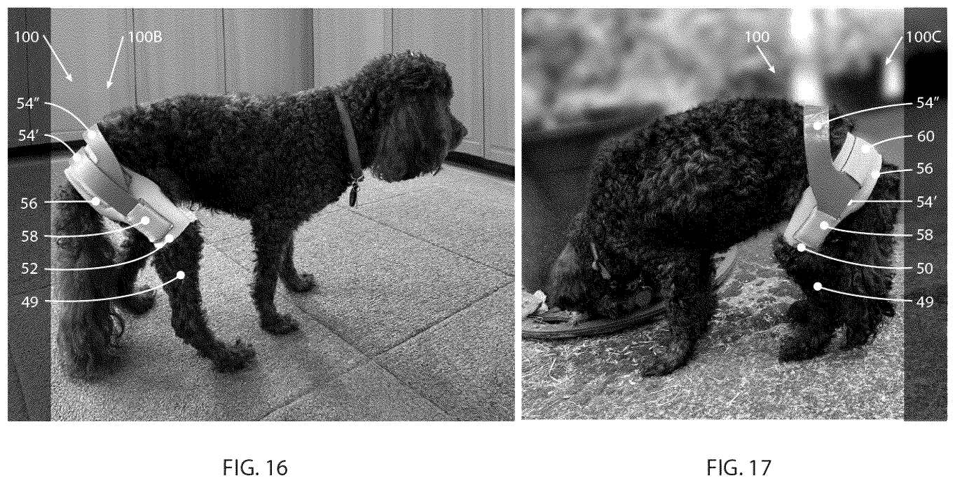

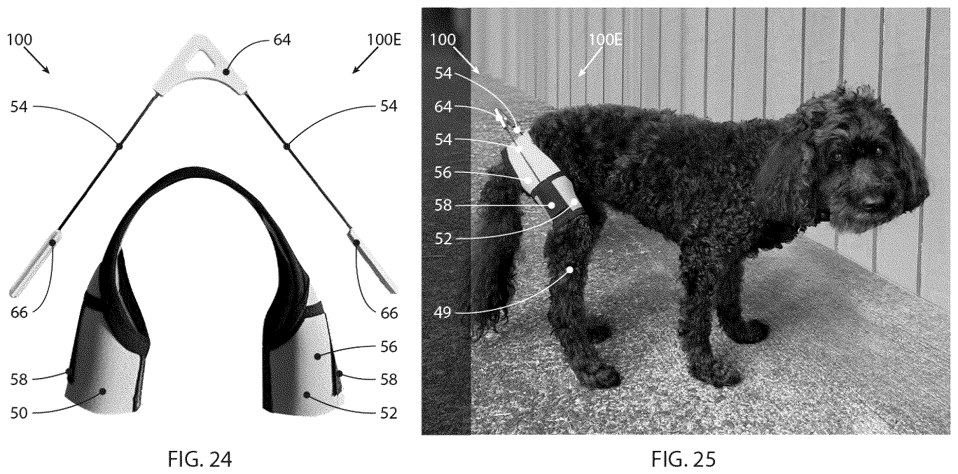

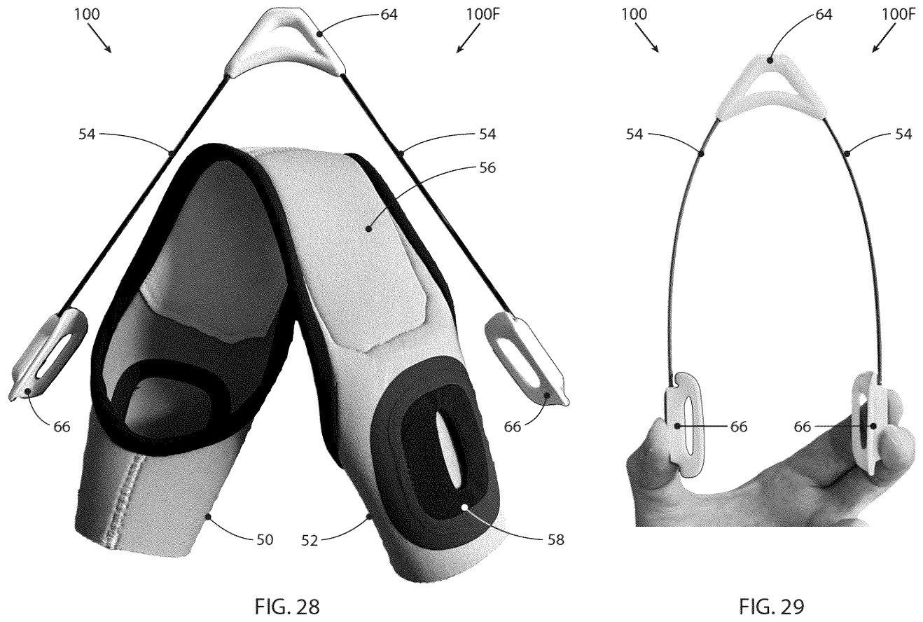

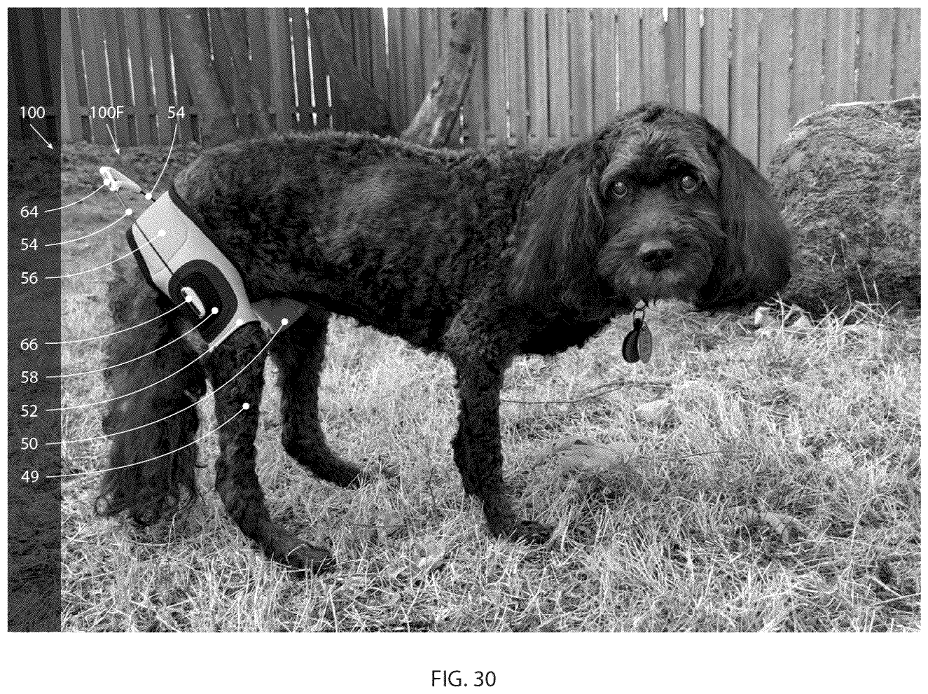

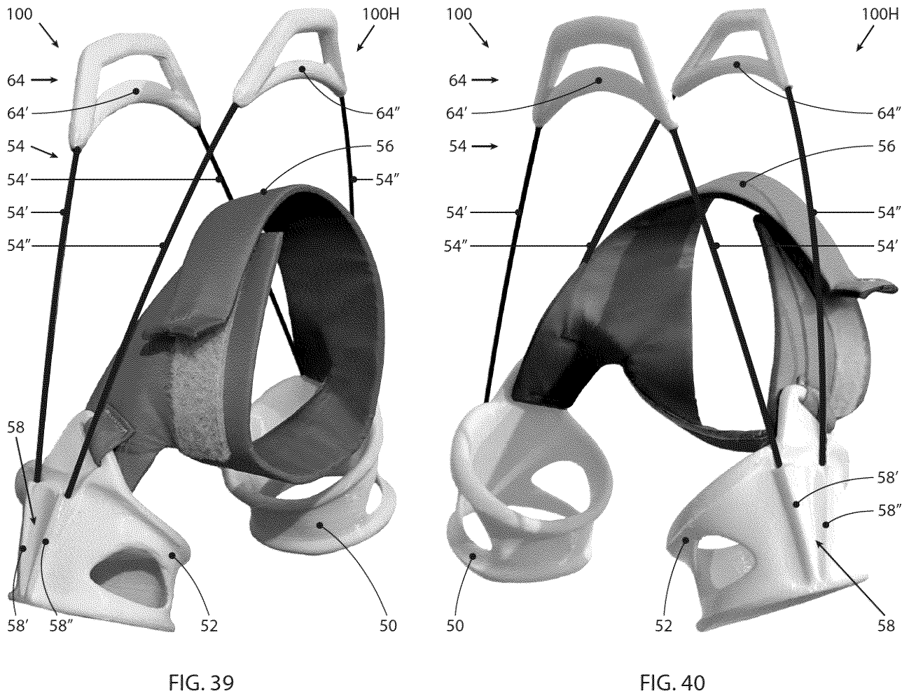

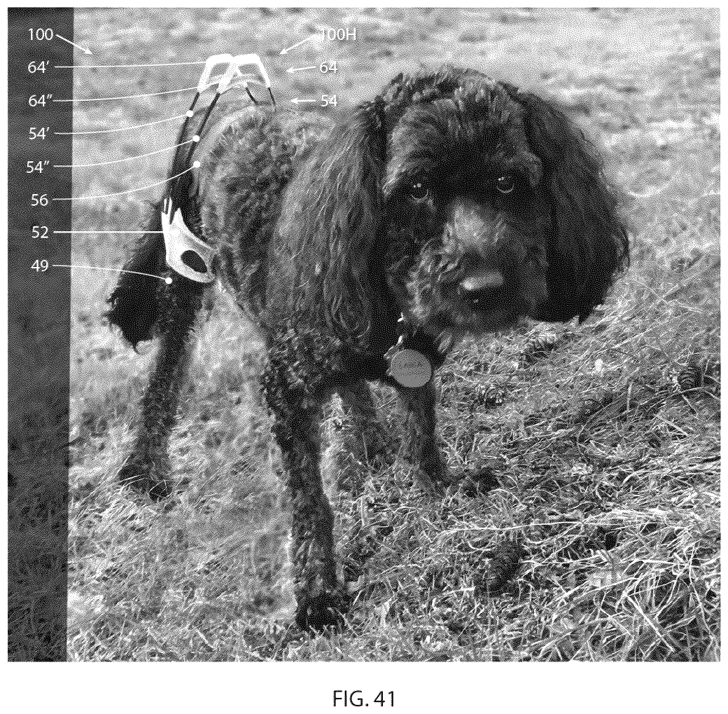

Referring to FIGS. 7-47, a dynamic joint alignment brace 100 for an animal, such as a canine 49, is shown that has at least one resilient member 54 deflected from is neutral position when operably secured to the leg of an animal to urge an animal's joint engagement with that leg toward a more desirable orientation without unduly compromising the freedom of movement of the animal. The dynamic joint alignment brace 100 may be particularly suitable for reducing symptoms associated with canine hip dysplasia (CHD) and may be suitable for reducing symptoms associated with other musculoskeletal conditions that may be common in animals with a skeletal structure including canines and the like. The dynamic joint alignment brace 100 applies a force which may persuade a canine's leg(s) in a direction that improves alignment of the leg bone(s) at the affected joint(s) throughout the range of motion of the leg(s). Exemplary embodiments of the dynamic joint alignment brace 100 will be described with reference to the various figures forming a part of the present disclosure.

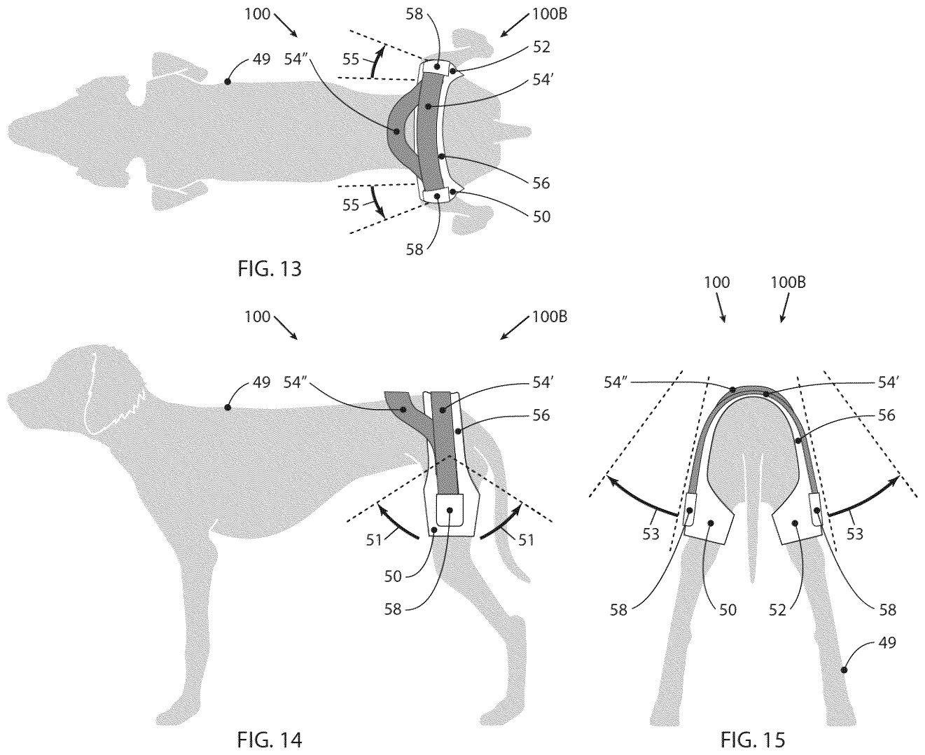

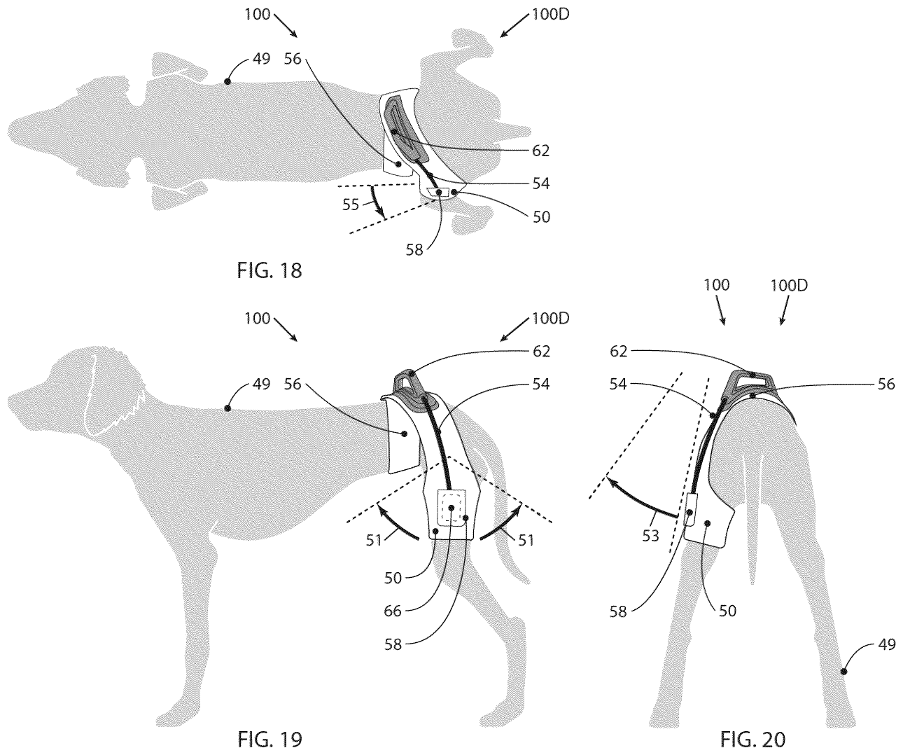

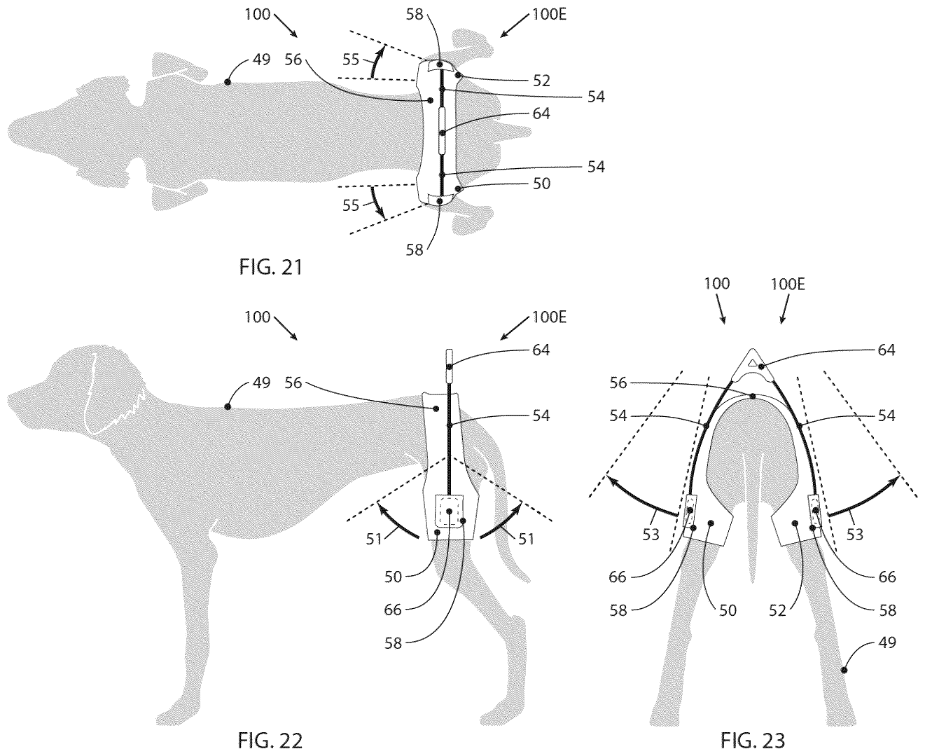

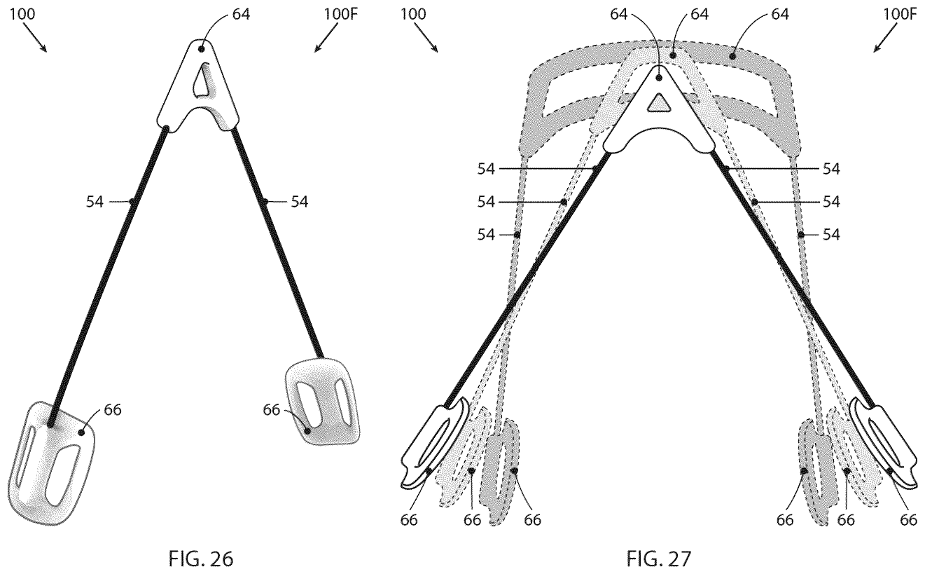

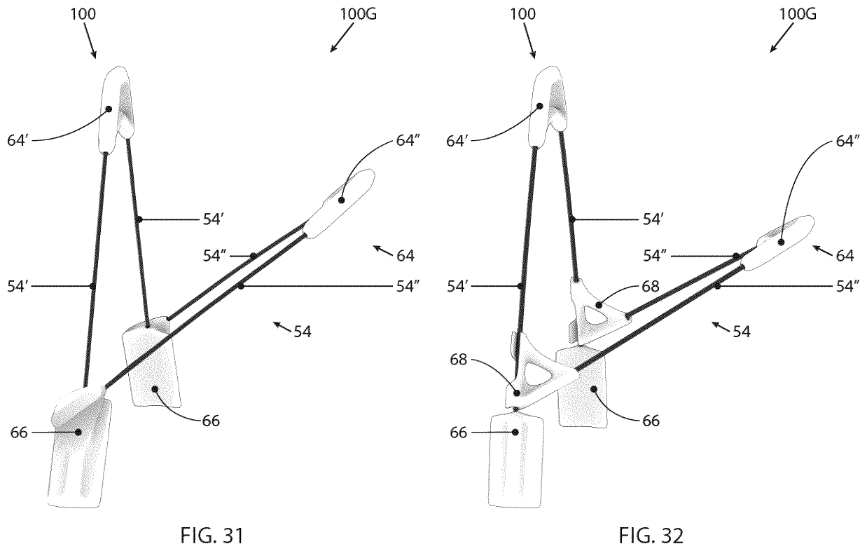

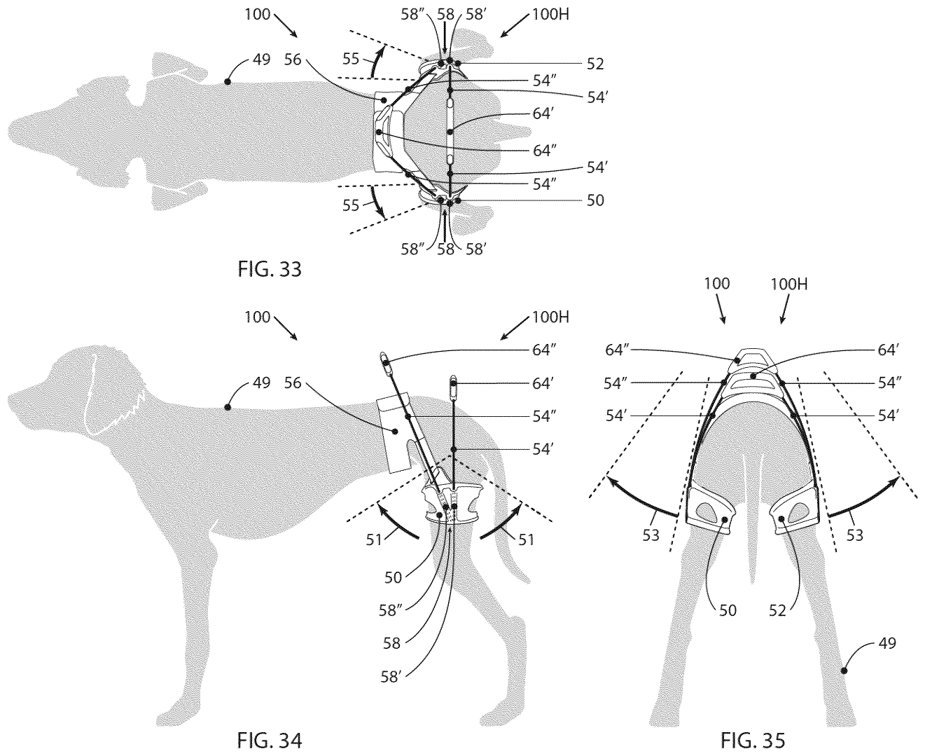

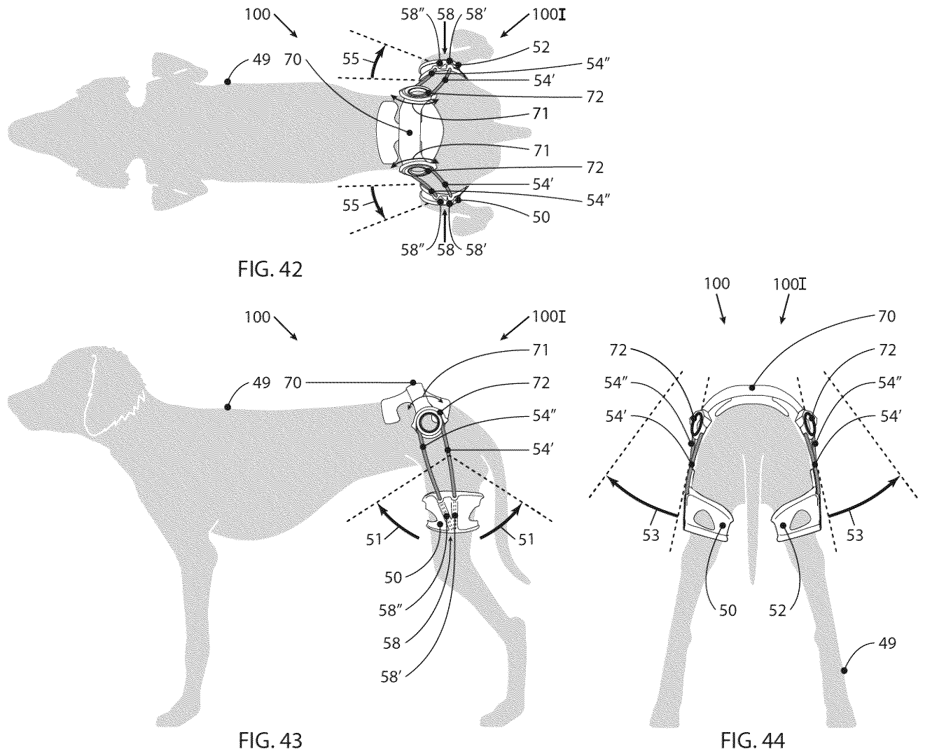

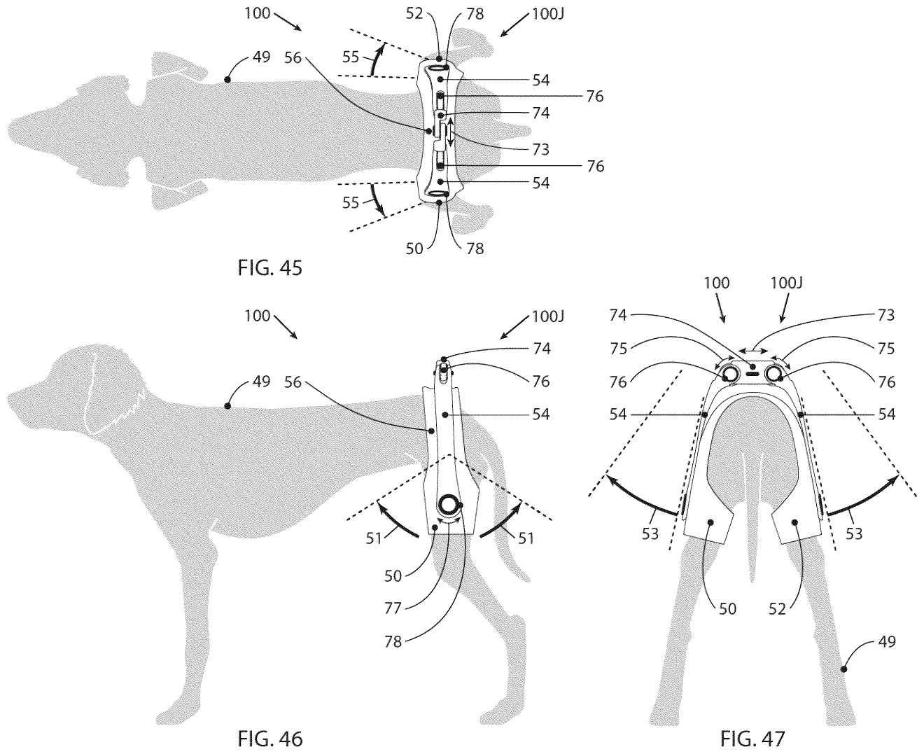

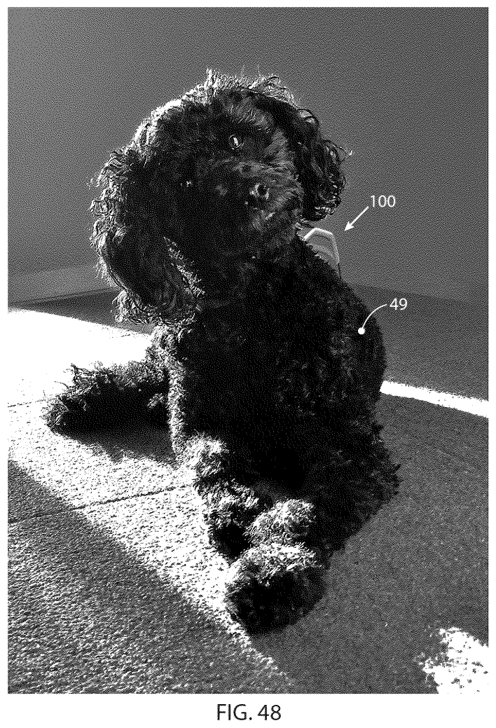

A first possible embodiment 100A of the dynamic joint alignment brace 100 is shown in FIGS. 7-12. A second possible embodiment 100B of the dynamic joint alignment brace 100 is shown in FIGS. 13-16. A third possible embodiment 100C of the dynamic joint alignment brace 100 is shown in FIG. 17, and a fourth possible embodiment 100D of the dynamic joint alignment brace 100 is shown in FIGS. 18-20. A fifth possible embodiment 100E of the dynamic joint alignment brace 100 is shown in FIGS. 21-25, and a sixth possible embodiment 100F of the dynamic joint alignment brace 100 is shown in FIGS. 26-30. A seventh possible embodiment 100G of the dynamic joint alignment brace 100 is shown in FIGS. 31-32. An eighth possible embodiment 100H of the dynamic joint alignment brace 100 is shown in FIGS. 33-41. A ninth possible embodiment 100I of the dynamic joint alignment brace 100 is shown in FIGS. 42-44. A tenth possible embodiment 100J of the dynamic joint alignment brace 100 is shown in FIGS. 45-47. In order to avoid undue repetition, like elements between the embodiments have like reference numbers.

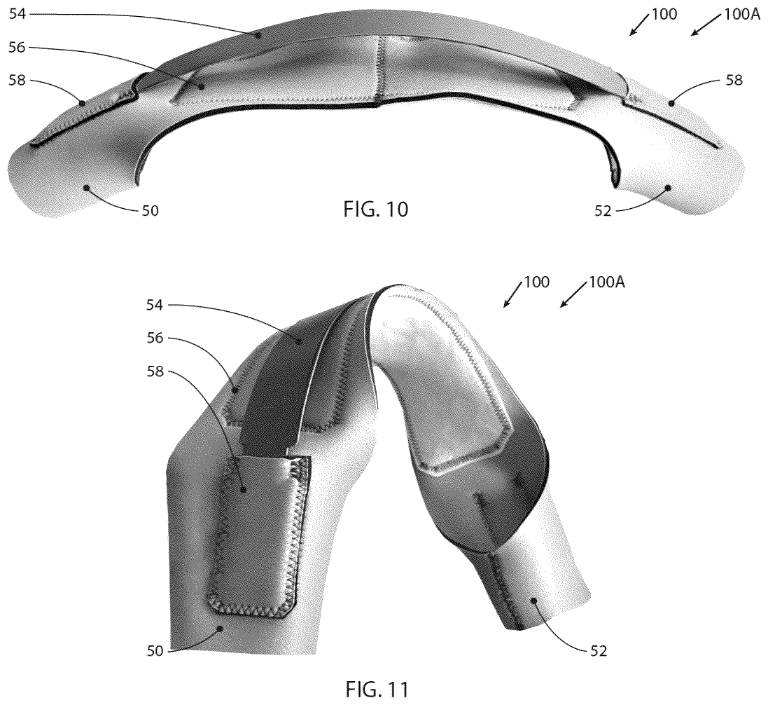

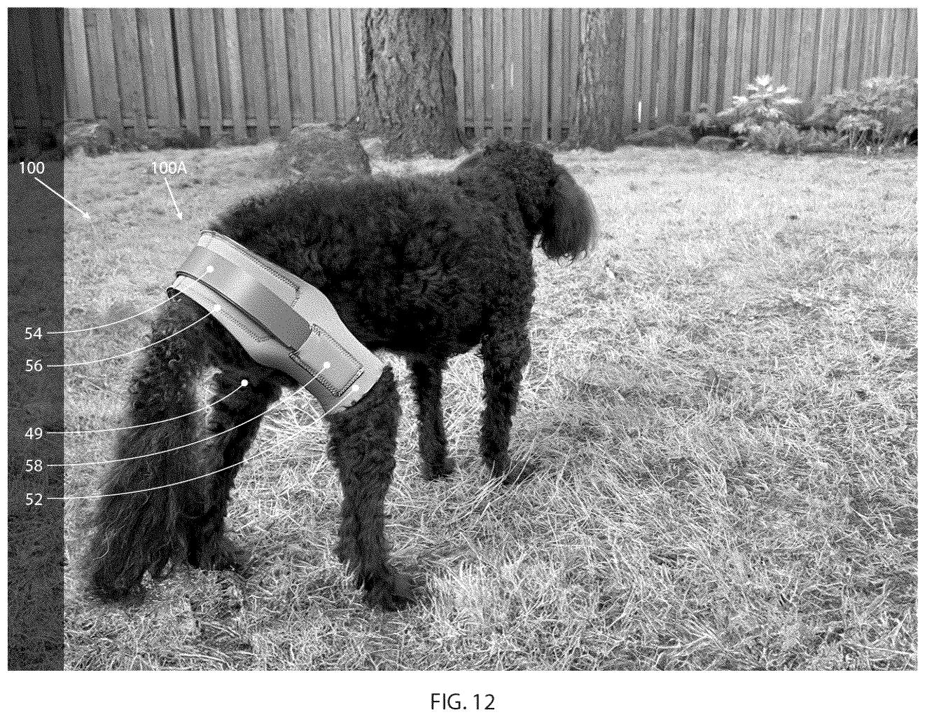

As shown in FIGS. 7-12, a first possible embodiment 100A of the dynamic joint alignment brace 100 is illustrated in top view in FIG. 7, in left side view in FIG. 8, in rear view in FIG. 9 as the various components are arranged in use. Additionally, this first possible embodiment of a dynamic joint alignment brace 100 is shown in FIGS. 10-11, and as applied to a canine 49 in FIG.12.

A dynamic joint alignment brace 100, represented in FIGS. 7-9, may include a superior harness 56 which may be detachably secured to a left rear leg encircling object 50 and a right rear leg encircling object 52 and may function to hold a left rear leg encircling object 50 and a right rear leg encircling object 52, each having a receptacle 58, such as a pocket, formed recess within the material, or the like, positioned at a proper height to operably, and preferably detachably, receive the distal ends of the resilient member 54 therein thereby holding the resilient member 54 in a position deflected from its neutral position when the superior harness 56 is secured to the canine 49.

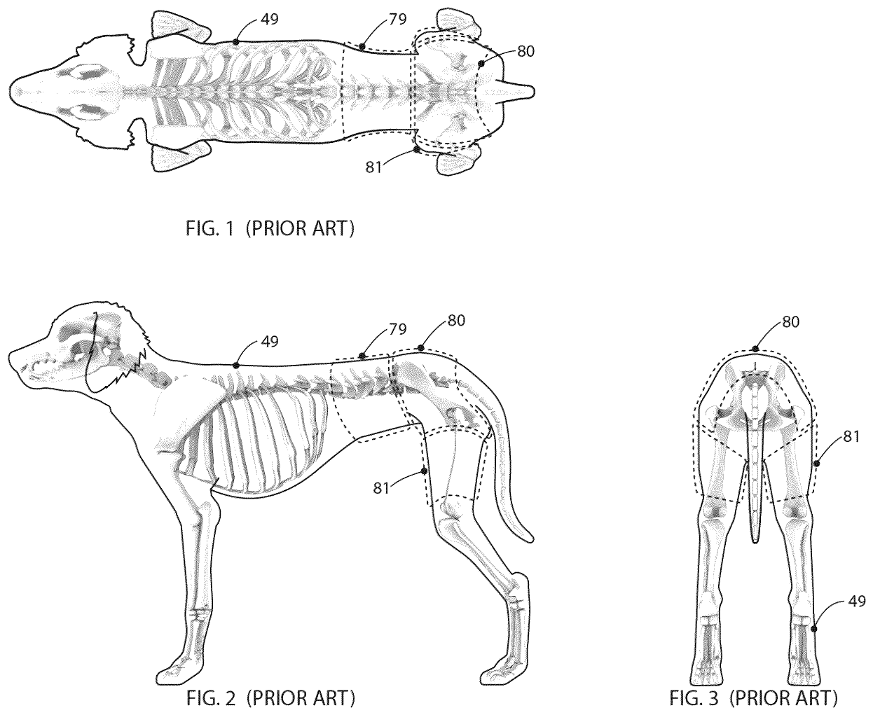

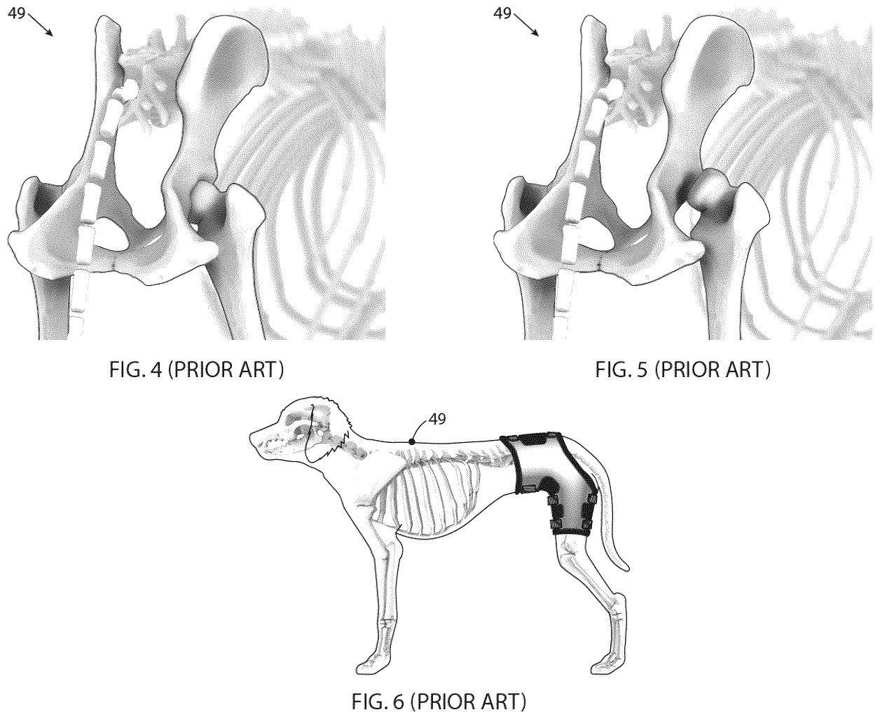

As shown in FIGS. 7-12, a superior harness 56 and a left rear leg encircling object 50 and a right rear leg encircling object 52 may be unified as a singular assembly. A superior harness 56 may follow the contour of the dorsal aspect of lumbar region 79 (FIGS. 1 & 2 (PRIOR ART)) of a canine. A left rear leg encircling object 50 and a right rear leg encircling object 52 may encircle the thigh region 81 (FIGS. 1-3 (PRIOR ART)) of a canine and may include a receptacle 58, such as a pocket, located on the lateral side of thigh region 81 (FIGS. 1-3 (PRIOR ART)). A superior harness 56 may be made of any material with particular attention paid to areas that may chafe and or cause discomfort. For example, superior harness 56 may be made of a soft resilient material such as neoprene with a hook and loop convention for donning and removing. Or, superior harness 56 may be made of a non-textile type material such as molded plastic with some level of added cushioning and may be detachably secured with durable hardware.

Referring to FIGS. 10-11, a resilient member 54 is shown that is preferably made of flexible, resilient material, such as plastic or the like. The resilient member 54 preferably has a neutral position as best shown in FIG. 10 that biased from its neutral position as shown in FIG. 11 when operably secured between the animal's body and leg to provide a useful force that urges the animal's leg in a desirable direction relative to an animal's joint. Resilient member 54 may be a single continuous piece made of one material. Cutting a sheet of a resilient material into a straight strip and deflecting it into a non-neutral position may create a force that is perpendicular to the surface of resilient member 54. An advantage of using a single continuous piece may be improved long-term durability, simple assembly and low-cost replacement. Resilient member 54 may be made of any material. An exemplary material for use, as shown in FIGS. 7-12, may include a sheet-based polymer, wooden material, a polymer-based extrusion such as acetal, carbon fiber sheeting and the like.

The dynamic joint alignment brace 100, represented in FIGS. 7-9, may include a left rear leg encircling object 50 and a right rear leg encircling object 52, which may direct the force created by resilient member 54 when resilient member 54 is biased in a non-neutral position to persuade the leg(s) laterally, a movement of the limb(s) away from the median plane and along the transverse plane of a canine wherein a canine's stance may be wider, abduction 53, referenced by the directional arrows associated therewith, as shown in FIG. 9. Preferably, this lateral force, abduction 53, as shown in FIG. 9, is applied throughout the forward and backward movement of the leg(s), along or parallel to the median plane of a canine, flexion and extension 51, referenced by the directional arrows associated therewith, as shown in FIG. 8, without excessively limiting the natural movements associated with walking.

As represented in the first possible embodiment 100A of the dynamic joint alignment brace 100, shown in FIGS. 7-12, may include a left rear leg encircling object 50 and a right rear leg encircling object 52, resilient member 54, and superior harness 56 as shown. The structure preferably persuades a canine's leg(s) laterally, away from the median plane of a canine, often referred to as abduction 53, and or it may rotate the leg(s) about the hip joint(s) away from the median plane and along the dorsal plane of a canine, often referred to as external rotation 55, referenced by the directional arrows associated therewith, as shown in FIG. 7. The movements abduction 53 and external rotation 55 may consequently persuade the femoral head to rotate in the acetabulum in such a way that malformed and or worn articular surfaces may be held in an augmented and or engaged position wherein the hip joint(s) may subsequently be less likely to subluxate and or luxate without excessively limiting flexion and extension 51.

The advantages that may be provided by the dynamic joint alignment brace 100 such as the useful lateral force, abduction 53, and or the unrestricted forward and backward movements, flexion and extension 51, and or the lateral rotation of the legs about the hip joint(s), external rotation 55, may be of different amounts based on the specific needs of a canine in question and may be improved by adjusting various aspects of the dynamic joint alignment brace 100.

As best shown in FIGS. 7 & 8, superior harness 56 may be constructed to follow the contour of the dorsal aspect of pelvis region 80 (FIGS. 1-3 (PRIOR ART)) at a variety of angles to the hip joint(s) such that superior harness 56 may modify or adjust the angular bias of the useful force provided by resilient member 54 and consequently may modify or adjust abduction 53 and external rotation 55.

The receptacle 58 may be positioned at a variety of heights and or angles on the lateral side of superior harness 56 which encircles thigh region 81 (FIGS. 1-3 (PRIOR ART)) on both a left rear leg encircling object 50 and a right rear leg encircling object 52 such that the receptacle 58 may modify or adjust the angular bias of the useful force provided by resilient member 54 and consequently may modify or adjust abduction 53 and or external rotation 55.

By supporting and or directing a canine's leg(s) in this manner, a dynamic joint alignment brace 100 may counteract the tendency of the hip joint(s) with laxity to subluxate and or luxate and may consequently mitigate or halt degenerative arthritic changes of the hip joint(s) and may subsequently reduce or eliminate the joint pain associated with CHD and may accordingly improve a canine's quality of life and longevity.

The second possible embodiment 100B of the dynamic joint alignment brace 100, as shown in FIGS. 13-16, preferably includes a plurality of resilient members 54. The plurality of resilient members 54 is shown in the drawings wherein a first resilient member may be specified as resilient member 54′ (54 prime) and a second resilient member may be specified as 54″ (54 double prime). Resilient member 54′ and resilient member 54″ may be of similar or different resiliencies. By adjusting various aspects of resilient member 54′ and resilient member 54″, their individual resilience may be altered and may be used to optimize and adjust an angular bias of a left rear leg encircling object 50 and a right rear leg encircling object 52. This angular bias allows a canine's leg(s) to be rotated about the hip joint(s), as represented by a range of angles, external rotation 55, as shown in FIG. 13, as needed, to further optimize the position of a canine's leg(s) relative the hip joint(s). Fabricating a plurality of resilient members 54 in a plurality of geometries, may customize the force created by resilient members 54 in its biased position. For example, cutting a sheet of resilient material into a straight strip and deflecting it into a non-neutral position may create a force that is perpendicular to the surface of resilient member 54, however, cutting a sheet of resilient material into a curved or arching shape, may create a non-perpendicular force to the surface of resilient member 54 that is deflected in a useful way.

Referring to FIG. 17, the third possible embodiment 100C of the dynamic joint alignment brace 100 is shown. In this embodiment the resilient member 54′ may follow the contour of a superior harness 56 and may be detachably secured to a superior harness 56 via sleeve 60 on the dorsal side of superior harness 56, and receptacle 58 on the lateral side of both a left rear leg encircling object 50 and a right rear leg encircling object 52. A left rear leg encircling object 50 and a right rear leg encircling object 52 may be connected to one another via superior harness 56, forming a single unit. Resilient member 54″ may be included and may also the utilize sleeve 60 and the receptacle 58 and may be a different geometry than resilient member 54′ and may modify or adjust a lateral movement of the leg(s) away from the median plane of a canine, external rotation 55. A plurality of resilient members 54 may include multiple layers of identical geometries or multiple layers with different geometries to provide an improved ability to direct the forces created by resilient members 54 in its biased position.

Referring to FIGS. 18-20, the fourth possible embodiment 100D of the dynamic joint alignment brace 100 is shown. In this embodiment, resilient member 54 may provide a functional benefit to only one of a canine's legs. A superior harness 56 may follow the contour of the left lateral aspect of thigh region 81 (FIGS. 1-3 (PRIOR ART)) and may continue over the dorsal aspect of pelvic region 80 (FIGS. 1-3 (PRIOR ART)) and may encircle the lumbar region 79 (FIGS. 1 & 2 (PRIOR ART)) caudally and may be detachably secured with a hook and loop fastener for donning and removing. A superior harness 56 may include an attached superior lumbar component 62, which may be affixed to and centered on the dorsal side of superior harness 56 and superior harness 56 may be positioned on the dorsal aspect of pelvic region 80 (FIGS. 1-3 (PRIOR ART)) on a canine. The resilient member 54 may include inferior segment 66 and may be detachably secured to both an attached superior lumbar component 62 and a left rear leg encircling object 50 via receptacle 58. The dynamic joint alignment brace 100 may be made specifically for a left rear leg, as shown in FIGS. 18-20, or specifically for a right rear leg, or a dynamic joint alignment brace 100 may be one unit and may be configured to be worn either on a left rear leg or a right rear leg. The resilient member 54 may be a continuous piece of one resilient material or may include a connecting element inferior segment 66. An advantage of using smaller segments including connecting elements may be an improved ability to direct the forces created by resilient member 54 in its biased position. The resilient member 54 may be made of any material. An exemplary material for use, as shown in FIGS. 18-20, may include a polymer-based extrusion such as acetal rod, carbon fiber rod, carbon fiber sheeting and the like, with connecting elements such as injection molded or 3d printed plastic, titanium and the like, or resins and the like.

Referring to FIGS. 21-25, the fifth possible embodiment 100E of the dynamic joint alignment brace 100 is shown. In this embodiment, resilient member 54 may be made of smaller segments including connecting elements and may be detachably secured to a left rear leg encircling object 50 and a right rear leg encircling object 52. A left rear leg encircling object 50 and a right rear leg encircling object 52 may include receptacle 58 on the lateral surface of thigh region 81 (FIGS. 1-3 (PRIOR ART)). The assembly may include superior segment 64, resilient member 54, and inferior segment 66. The assembly may be detachably secured to a superior harness 56 via receptacle 58.

Referring to FIGS. 26-30, the sixth possible embodiment 100F of the dynamic joint alignment brace 100 is shown. In this embodiment, resilient member 54 may be detachably secured to a left rear leg encircling object 50 and a right rear leg encircling object 52. The resilient member 54 may include inferior segment 66 which may be fabricated to follow the contour of the lateral aspect of thigh region 81 (FIGS. 1-3 (PRIOR ART)) of a canine and may be held at a proper height between the hip and stifle of a canine, via receptacle 58. A left rear leg encircling object 50 and a right rear leg encircling object 52 may be connected to one another via superior harness 56, forming a single unit. Additionally, as shown in FIG. 27, superior segment 64 may be fabricated in various shapes and or sizes and may modify or adjust the useful force resilient member 54 may provide. As shown in FIG. 26, the triangular shape of superior segment 64 may create a stronger force, abduction 53, on the limb(s) of a canine in its biased position than the other examples of superior segment 64, as shown in FIG. 27. Spacing the two resilient member 54 sections farther apart and or positioning them at different angles to a canine's hip joint(s) may provide a range of customization and benefits to a specific canine.

Referring to FIGS. 31 & 32, the seventh possible embodiment 100G of dynamic joint alignment brace 100 is shown. In this embodiment, a plurality of resilient members 54 and a plurality of assemblies may further customize the useful force applied to the animal. A superior harness 56, as shown in FIGS. 7-16, FIGS. 21-25, FIG. 28, and FIG. 30, may be included.

As best shown in FIG. 31, a plurality of resilient members 54 may include a first assembly in which superior segment 64′ and resilient member 54′ may be affixed to inferior segment 66 at a specified angle and a second assembly in which superior segment 64″ and resilient member 54″ may be affixed to inferior segment 66 at a different specified angle.

Referring to FIG. 32, a variation of the seventh possible embodiment 100G is shown, wherein a plurality of resilient members 54 may include a first assembly in which superior segment 64′, resilient member 54′, and inferior segment 66, may be detachably secured and coupled to a second assembly which may include superior segment 64″. resilient member 54″, and coupling segment 68. Coupling segment 68 may be made in a plurality of angles to customize the useful force applied to the animal.

Referring to FIGS. 33-41, the eighth possible embodiment 100H of the dynamic joint alignment brace 100 is shown and represents the inventors' current preferred embodiment. The various elements in this embodiment, as they are arranged, may provide the same benefits as other embodiments in this disclosure and may include a plurality of resilient members 54 in which resilient member 54″ may be held at an angle pitched ˜25° forward from resilient member 54′, wherein the distal end of resilient member 54′ is the vertex of the angle measurement for placement of resilient member 54″.

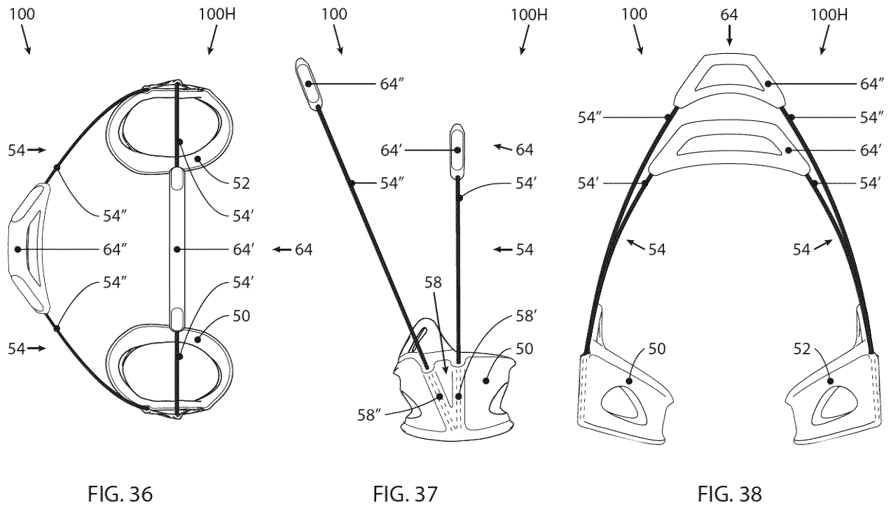

FIGS. 33-41 demonstrate the incorporation of soft thermoplastic parts created using additive manufacturing to replace previous fabric versions of a left rear leg encircling object 50 and a right rear leg encircling object 52. An advantage of using this type of part in the overall assembly is that it may be easily customized for a canine's specific needs. In FIGS. 36-40, it is demonstrated that special attention may be given to all aspects of a left rear leg encircling object 50 and a right rear leg encircling object 52 to ensure smooth interior surfaces and rounded edges curve outwardly, away from the thigh region 81 (FIGS. 1-3 (PRIOR ART)), thereby minimizing areas of friction where surface contact may be made with a canine and providing a wide range of movements without restriction or discomfort. A left rear leg encircling object 50 and a right rear leg encircling object 52 may include cutouts for breathability and may allow minimal surface area contact with thigh region 81 (FIGS. 1-3 (PRIOR ART)). A left rear leg encircling object 50 and a right rear leg encircling object 52 may be made of a translucent material and may allow the ability to monitor a canine's skin and fur and may consequently allow any skin chafing or discomfort to be detected immediately.

A plurality of resilient members 54 may be detachably secured to a left rear leg encircling object 50 a right rear leg encircling object 52 for donning and removing. A left rear leg encircling object 50 and a right rear leg encircling object 52 may include a plurality of receptacles 58, which are preferably recesses formed in the leg encircling objects 50, 52, for a plurality of resilient members 54, and may be detachably secured to superior harness 56, which may hold a left rear leg encircling object 50 and a right rear leg encircling object 52 at an optimal position on the lateral aspect of thigh region 81 (FIGS. 1-3 (PRIOR ART)), wherein the useful forces applied to the animal may be customized and may optimize abduction 53 and external rotation 55 without limiting flexion and extension 51.

A superior harness 56 may closely encircle the dorsal aspect and the ventral aspect of lumbar region 79 (FIGS. 1 & 2 (PRIOR ART)) of a canine and may be detachably secured and adjustably sized, for example, using a hook and loop fastener, to fit a specific canine without restricting circulation. The superior harness 56 may allow a range of movements wherein a canine may independently sit down, lay down, sleep, and stand up from any surface, and may also allow a canine to groom itself and perform its biological activities unencumbered, without the removal of the dynamic joint alignment brace 100.

Referring to FIGS. 43-44, the ninth possible embodiment 100I of the dynamic joint alignment brace 100 is shown. In this embodiment, resilient member 54′ and resilient member 54″ may be attached to superior hinge 72. Superior hinge 72 may be attached to superior lumbar component 70, which may rest on the dorsal aspect of lumbar region 79 (FIGS. 1 & 2 (PRIOR ART), and may minimize surface area contact with a canine. Resilient member 54′ and resilient member 54″ may be detachably secured to a left rear leg encircling object 50 and a right rear leg encircling object 52. A left rear leg encircling object 50 and a right rear leg encircling object 52 may pendulate on superior hinge 72, pendulation 71, and may allow flexion and extension 51. Superior hinge 72 may also include adjustable stops which may allow a specified and finite range of motion, pendulation 71, wherein incremental therapeutic strengthening may be beneficial to a canine.

Referring to FIGS. 45-47, the tenth possible embodiment 100J of the dynamic joint alignment brace 100 is shown. In this embodiment, resilient member 54 may be held in place by superior indexing segment 74, which may be fully adjustable and customizable to fit different sized canines. Superior indexing segment 74 may be adjustable in width, in the direction of arrows associated with lateral adjustment 73. Additionally, indexing angular joints 76 may be included, which may serve to provide adjustability to the respective angle resilient members 54 in their unbiased position to define an angle articulation range 75 about each angular joint 76 as best shown in FIG. 47. These structures allow the dynamic joint alignment brace 100 to fit and provide the appropriate useful force of abduction 53 for a range of differently sized canines.

An additional feature of the tenth possible embodiment 100J may be the ability of a left rear leg encircling object 50 and a right rear leg encircling object 52 to freely rotate and or be fixed at a series of orientations within a range about inferior pivot 78 in the direction of the arrows associated with rotation 77. Resilient member 54 may be pivotally and or detachably secured about inferior pivot 78 to a left rear leg encircling object 50 and a right rear leg encircling object 52 allowing for some freedom of articulation within the assembly, and, if preferred, inferior pivot 78 may be able to be locked into a position.

While the subject disclosure has been described with reference to exemplary embodiments, it will be appreciated by those skilled in the art that various changes may be made and equivalents may be substituted for elements thereof without departing from the scope of the subject disclosure. For example, the invention has been described with particular emphasis on the preferred embodiments and with a focus on addressing issues related to CHD. However, other musculoskeletal conditions that may be common in animals with a skeletal structure including canines and the like, may benefit from the invention. It would be realized from the teachings herein that other embodiments, alterations, configurations and related uses could be employed without departing from the scope of the invention. In addition, modifications may be made to adapt a particular situation or material to the teachings of the exemplary embodiments without departing from the essential scope thereof.

Moreover, directional terms such as top, bottom, left, right, above, below and diagonal are used with respect to the accompanying drawings. The term “proximal” shall mean closer to the spine or closer to the origin of a limb. The term “distal” shall mean farther away from the spine or farther away from the origin of a limb. The term “medial” shall mean the middle or direction toward the middle or direction toward the median plane. The term “lateral” shall mean the side or direction toward the side or direction away from the median plane. The terms “flexion” and “extension” shall mean forward and backward movement of limb that happen along or parallel to the median plane. The term “adduction” shall mean movement of a limb towards the midline of the body along the transverse plane. The term “abduction” shall mean movement of a limb away from the midline of the body along the transverse plane. The term “internal rotation” shall mean rotation of a limb towards the center of the body along the dorsal plane. The term “external rotation” shall mean rotation of a limb away from the center of the body along the dorsal plane. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any matter not explicitly set forth. Additionally, the term “a,” as used in the specification, means “at least one.” The terminology includes the words above specifically mentioned, derivatives thereof, and words of similar import.

Throughout this disclosure, various aspects of the subject disclosure can be presented in a range format. It should be understood that the description in range format is merely for convenience and brevity and should not be construed as an inflexible limitation on the scope of the exemplary embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the exemplary embodiments can be practiced without one or more of the specific features or advantages of a particular embodiment. In other instances, additional features and advantages may be recognized in certain embodiments that may not be present in all exemplary embodiments of the subject disclosure. It is to be understood, therefore, that the exemplary embodiments are not limited to the particular aspects disclosed, and they are intended to cover modifications within the spirit and scope of the subject disclosure as defined by the appended claims.

1. A dynamic joint alignment brace for aligning an animal's leg to a joint comprising:
a detachable mounting portion sized to operably engage the animal's leg;
a resilient member operably secured to the detachable mounting portion and biased to a neutral position; and,
the resilient member deflected from its neutral position when the detachable mounting portion is operably secured to the animal's leg such that the resilient member applies a biasing force that urges the animal's leg into a desired alignment position relative to the animal's joint.

2. The dynamic joint alignment brace of claim 1, wherein the desired alignment position is abduction.

3. The dynamic joint alignment brace of claim 1, further including:
a second detachable mounting portion, the first detachable mounting portion operably secured to an animal's first leg, and the second detachable mounting portion operably secured to the animal's second leg; and,
the resilient member extending between the first detachable mounting portion and the second detachable mounting portion.

4. The dynamic joint alignment brace of claim 3, further including a second resilient member biased to a second neutral position extending between the first detachable mounting portion and the second detachable mounting portion such that the second resilient member is deflected from the second neutral position thereby applying a second biasing force to the animal.

5. The dynamic joint alignment brace of claim 4, wherein the biasing force of the first resilient member is different from the second biasing force of the second resilient member.

6. The dynamic joint alignment brace of claim 5, wherein the desired alignment position is a combination of abduction and external rotation about the animal's joint.

7. The dynamic joint alignment brace of claim 1, wherein the animal is a canine and the animal's joint is a hip joint.

8. The dynamic joint alignment brace of claim 1, wherein the resilient member is substantially planar when in its neutral position.

9. The dynamic joint alignment brace of claim 1, wherein the resilient member is a flexible elongate rod.

10. The dynamic joint alignment brace of claim 1, wherein the detachable mounting portion includes a left rear leg encircling object and a right rear leg encircling object, and the resilient member operably extends between the left rear leg encircling object and right rear leg encircling object when the resilient member is deflected from its neutral position.

11. The dynamic joint alignment brace of claim 10, wherein at least one of the left rear leg encircling objects and right rear leg encircling objects is selected from the group consisting of fabric, molded material, and soft thermoplastic created using additive manufacturing.

12. The dynamic joint alignment brace of claim 1, further including a harness encircling the pelvis region of the animal, the harness operably connected to the resilient member to hold the resilient member in place on the animal when the resilient member operably engages the leg of the animal.

13. The dynamic joint alignment brace of claim 12, wherein:
the detachable mounting portion includes a left rear leg encircling object and a right rear leg encircling object;
the resilient member operably extends between the left rear leg encircling object and right rear leg encircling object when the resilient member is deflected from its neutral position;
the resilient member has a first distal end and an opposite second distal end; and,
the dynamic joint alignment brace further including:
a first receptacle connected to the right rear leg encircling object;
a second receptacle connected to the left rear leg encircling object; and,
whereby the first distal end of the resilient member is operably received within the first receptacle and the opposite second distal end of the resilient member is operably received within the second receptacle.

14. The dynamic joint alignment brace of claim 13, wherein one of the first and second receptacles is selected from the group consisting of a pocket secured on, an opening formed in, and a recess formed in one of the left and right rear leg encircling objects.

15. The dynamic joint alignment brace of claim 1, further including a harness detachably encircling the lumbar region of the animal, the harness operably connected to at least one left rear leg encircling objects and right leg encircling objects to operably hold the at least one left and right leg encircling objects in place on the animal.

16. The dynamic joint alignment brace of claim 1, wherein the resilient member is pivotally secured to the animal's leg, thereby facilitating flexion and extension movement of the animal's leg without compromising the biasing force that urges the animal's leg into a desired alignment position relative to the animal's joint.

17. The dynamic joint alignment brace of claim 1, wherein the resilient member includes a first resilient portion, a second resilient portion and a lumbar component extending between the first and second resilient portions. The lumbar component has a defined width sized to extend across the lumbar region of the animal when the dynamic joint alignment brace is operably secured to the animal.

18. The dynamic joint alignment brace of claim 17, further including an adjustment structure to allow the defined width of the lumbar component to be adjusted.
